(19) United States
(12) Patent Application Publication
(10) Pub. No.: US 2024/0065830 A1
(43) Pub. Date: Feb. 29, 2024

(54) MECHANICAL VALVE SYSTEMS WITH IMPROVED PROPERTIES

(71) Applicant: Michael COHEN, Gush Etzion (IL)

(72) Inventor: Michael COHEN, Gush Etzion (IL)

(21) Appl. No.: 18/261,098

(22) PCT Filed: Jan. 12, 2022
(86) PCT No.: PCT/IL2022/050048

§ 371 Date: Jul. 11, 2023

Related U.S. Application Data
(60) Provisional application No. 63/136,657, filed on Jan. 13, 2021.

(51) Int. Cl. A61F 2/24

(52) U.S. Cl. CPC A61F 2/2403; A61F 2210/009

(57) ABSTRACT

This invention provides mechanical valve device and ring systems for cardiac valve replacement, wherein the mechanical valve devices comprises two or more leaflets, each leaflet comprising at least a first material which reduces electrostatic forces between blood and device components, reduces adhesion between blood and device components or a combination thereof; and optionally further comprises at least a first magnet incorporated within at least one of the leaflets; and a valve constraining ring, comprising at least a first material which reduces electrostatic forces between blood and device components, reduces adhesion between blood and device components or a combination thereof; and internally located attachment regions for the leaflets; and optionally at least a second magnet of matched polarity to said first magnet, incorporated within the valve constraining ring at regions proximal to said first magnet within said leaflet such that repulsive forces are present when said first magnet and said second magnet are brought into proximity with each other in the absence of flow; and one or more extending projections, which reduce turbulent flow through the device, or proximally thereto, which are located on the leaflets of the valve device, or which are located on the constraining ring component of the device or a combination thereof. Uses of the valve and ring systems as described are provided as well.

### TECHNICAL FIELD

The present disclosure relates to a mechanical valve and ring systems for cardiac valve replacement. Some of the features of the valve and ring systems include a construction and geometry that reduce the incidence and/or magnitude of valve-related thrombosis. Other features of the valve and ring systems include combining materials that reduce electrostatic forces between blood components, and/or reduces adhesion of blood components to components of the valve systems of this invention and optionally reduces turbulent flow through the valve and/or tissue located proximally thereto, each feature of which, in turn, may reduce valve-related thrombosis.

### BACKGROUND OF THE INVENTION

Heart valve disease is a condition in which one or more of the cardiac valves of a patient malfunction. In adults, valvular heart disease continues to be a major cause of morbidity and mortality, usually due to valve stenosis, insufficiency or a combination of the two.

There are typically two types of prosthetic heart valves used to replace native diseased valves, namely mechanical and bioprosthetic valve replacements. Generally, mechanical heart valves are made entirely of synthetic materials, such as metals and pyrolytic carbon polymers. Bioprosthetic heart valves are made of either bovine pericardium or porcine aortic valves but may also be produced from equine or porcine pericardium. Human valves from donor hearts (homografts) can be used as well. Mechanical heart valves are very durable and may last decades.

One of the major drawbacks of mechanical heart valves is the requirement for daily anticoagulant warfarin due to an increased risk of artificial material induced thrombosis and thromboembolism. Valve related complications of mechanical valves include thromboembolisms, bleeding, patient prosthesis mismatch, endocarditis and others. Bio-prosthetic heart valves have improved central blood flow due to their bio-mimicking of tri-leaflet design (although regarding human mitral valves, with two leaflets there is no comparable biological valve suitable for same using xenografts). Use of bio-prosthetic valves does not generally require anticoagulant therapy. However, bio-prosthetic heart valves also have some drawbacks including limited durability due to structural valve degeneration—leaflet calcification, leaflet tearing, fatigue damage, and tissue failure. Therefore, 20 to 40 percent of homograft bio-prostheses and 20 percent of xenograft bio-prostheses fail within 10 to 15 years of implantation and require re-replacement. Furthermore, each valve replacement surgery increases the likelihood of adverse events including mortality.

There is a need for improved heart valve systems, which provide the benefits of the bio-prosthetic heart in terms of the lack of a need for sustained anti-coagulant therapy, which as well, provides the longevity of the mechanical heart valves and improved hemodynamic parameters.

### SUMMARY

It is an object of this invention to provide an improved mechanical heart valve system, which reduces the incidence and/or magnitude of valve-related thrombosis while improving a hemodynamic profile.

It is a further object of this invention to provide an improved heart valve system that has features combining materials that reduce electrostatic forces between blood and valve components, and/or reduces adhesion of blood components to said valve components and optionally reduces turbulent blood flow through the device or proximal to tissue located nearby, each feature of which may contribute to reduced valve-related thrombosis.

The valve systems of this invention will be comprised, in some embodiments, of a material which functions to reduce electrostatic forces between blood and valve components, and/or reduce adhesion of blood components to the valve device components or a combination thereof in the devices of this invention or tissue located proximally thereto.

In some aspects, the valve is comprised of a composite of at least a first material and at least a second material.

In some embodiments, the reference to the term “composite” may refer to the fact that two different materials are incorporated in a given part of the valve systems as herein defined. In some aspects, the reference to the term “composite” may refer to the fact that two different materials are incorporated on two different regions or parts of the valve system as herein defined. In some embodiments, the reference to the term “composite” may refer to a first material being a coating as applied onto the second material, or vice versa. The reference to the term “composite” may refer to the fact that two different materials are incorporated in a given part of the valve systems as herein defined, however, one of the materials is removed or selectively excluded, for example by evaporation or other means so that the final product may comprise only a single material that reduces electrostatic forces between blood components, and/or reduce adhesion of blood components to said valve and/or ring devices or a combination thereof and optionally reduces turbulent blood flow proximally to the device or tissue upstream or downstream of same.

In some embodiments the materials of which valve and ring systems of this invention will be comprised are milled and polished to a very high smoothness.

In some aspects, the valve systems of this invention will be comprised of multiple different layers with each layer being comprised of a material as described herein. In some embodiments, the valve systems of this invention will be comprised of any appropriate composite arrangement of materials, which impart the characteristics as descried herein.

In some aspects, the composite nature of the valve systems of this invention will include such composite feature being incorporated in particular regions of the valve device, whereby a first material, such as a ceramic material, which functions to reduce electrostatic forces between blood and valve components and/or reduce adhesion of blood components to said valve device or a combination thereof, for example, is present on certain regions of the valve device, and a second material, which may provide other advantages or serve as a biocompatible framework, or also functions to reduce electrostatic forces between blood and valve components and/or reduce adhesion of blood components to said valve device or a combination thereof may be located on different regions of the valve and/or ring device. In some embodiments the biocompatible ceramic material is a coating on an underlying substrate framework for the mechanical valve. In some embodiments the mechanical valve is comprised entirely of a biocompatible ceramic material or a composite of biocompatible ceramic materials.

According to this aspect, and in one embodiment, the second material may be of a different material than the first material, but possess very similar characteristics as the first material. According to this aspect, and in another embodiment, the second material may be of a different material than the first material, and possess different characteristics from the first material.

Still according to this aspect, and in another embodiment, the second material may be of an identical material to the first material, in other words, the components of the material are identical, and in some embodiments, the thickness, or coating orientation, differs from the first material.

In another embodiment, the first and second materials are identical in entirety.

In some aspects, when a composite material is used in the valve systems of this invention, the composite nature provides the versatility of a surface exposed layer possessing the properties of functioning to reduce electrostatic forces between blood and valve components and/or reduce adhesion of blood components to said valve device or a combination thereof, whereas a second material, which will also be biocompatible, may impart other advantages, or be inert and merely function as a good substrate for the support of the first material.

In some aspects, the composite nature of the valve devices of this invention will include such composite feature being incorporated in particular regions of the valve and/or ring device, whereby a first material, which functions to reduce electrostatic forces between blood components and/or reduce adhesion of blood components to said valve and/or ring devices or a combination thereof and/or a second material, functions to reduce electrostatic forces between blood and valve components and/or reduce adhesion of blood components to said valve and/or ring devices or a combination thereof may be located on the same regions of the valve, and in some embodiments, the materials may be located side-by-side, or in some aspects, as layers, where one layer is more surface exposed than another layer.

According to this aspect, and in some embodiments, the first material may be located on surface exposed regions of the leaflets of the claimed device, while the second material, may be located underneath the first layer. In some embodiments, the material may be located on one surface of the leaflet of a valve device of this invention, while the second material may be located on the opposite surface of the leaflet of a valve device of this invention. In some embodiments, where the valve devices of this invention may comprise two or more leaflets, the orientation of placement of the first and second materials, with respect to the surface exposure on a given leaflet may be constant, i.e. if there are two leaflets, then both leaflets will comprise the first material on a first surface of the leaflet and the second material on the opposing surface of the leaflet. In some embodiments, the orientation of placement of the first and second materials may be alternated with respect to the surface exposure on a given leaflet, such that one leaflet will comprise the first material on a first surface of the first leaflet and the second material on the first surface of the second leaflet, and the second surface of the first and second leaflet will similarly be comprised of the second and first materials, respectively. It will be appreciated that such patterns can be adapted, depending on the number of leaflets in the valve device and all such patterning is to be considered as part of the invention.

In some aspects the valve devices of this invention specifically regulate the blood flow through the device, to reduce turbulent flow. In some embodiments, the valve devices of this invention surprisingly achieve the ability to modulate blood flow to approximate laminar flow.

In some aspects, the valve devices of this invention specifically regulate the blood flow through the device to promote or approximate linear flow through a main orifice of the device, and in some aspects, regulate non-linear or turbulent flow through other areas of the valve device, for example, on the valve device sides or hinge regions.

In some aspects the valve devices of this invention specifically regulate the flow through the device, such that turbulent flow is reduced.

In some aspects, the valve devices of this invention provide for convex or concave positioning of the valve leaflets, which, in some aspects may be fully rotated to alter the positioning from convex to concave and vice versa.

In some aspects, the valve device may comprise an attachment section around the outer rim of the device, to promote fast and secure positioning and locking of same. In some aspects, such attachment section may include a ring comprised of synthetic or other suitable material, such as a Dacron ring such as from Dacron tube grafts (Vascutek, Terumo, Japan) or polytetrafluoroethylene (PTFE) suture annuloplasty is contemplated for such purpose.

In some aspects, the valve device may comprise ratcheting locking mechanisms around the outer rim of the device, to promote fast and secure positioning and locking of same.

The invention therefore provides, in some embodiments, a valve device, comprising:
two or more composite leaflets; comprising:
   at least a first material which reduces electrostatic forces between blood components and leaflet components and/or reduces adhesion of blood components to said valve device or a combination thereof; and
   optionally at least a second material, which reduces electrostatic forces between blood components and leaflet components and/or reduces adhesion of blood components to said valve device or a combination thereof; and
   optionally at least a first magnet incorporated within said composite leaflet structure; and
a valve constraining ring, comprising:
   at least a first material which reduces electrostatic forces between blood components and valve constraining ring components and/or reduces adhesion of blood components to said valve device or a combination thereof; and
   optionally at least a second material, which reduces electrostatic forces between blood components and valve constraining ring components and/or reduces adhesion of blood components to said valve device or a combination thereof; and
   internally located attachment regions for said two or more composite leaflets; and
   optionally at least a second magnet of matched polarity to said first magnet, incorporated within the valve constraining ring at regions proximal to said first magnet within said leaflet such that repulsive forces are present when said first magnet and said second magnet are brought into proximity with each other in the absence of flow; and
one or more extending projections, which reduce turbulent flow through the device, or proximally thereto, which are located on the leaflets of the valve device, or which are located on the constraining ring component of the device or a combination thereof.

In some embodiments, one or more drainage ports are located at a junction point between the composite leaflets and valve constraining ring.

This invention also provides methods of use of the valve devices of this invention for replacement or repair of a defective or non-functional valve in a subject in need thereof, which method provides for reducing the need to administer anti-thrombotic therapy to the patient undergoing or having undergone a valve replacement procedure.

In some embodiments, the term “reducing the need to administer anti-thrombotic therapy” will be reflected in any reduction in an anti-thrombotic treatment regimen, including number of dosages delivered over time, concentration or a combination thereof. In some embodiments, the framework for “reducing the need to administer anti-thrombotic therapy” will be a reflection of the dosage regimen that would have been arrived at for a given patient, based on various clinical indicators and established criteria such that the “reducing the need to administer anti-thrombotic therapy” will be any reduction in what would have ordinarily been the treatment regimen for the same subject implanted with a mechanical valve representing the current standard of care.

In another object of this invention there is provided an improved modular heart valve, which is so constructed so as to reduce the incidence and/or magnitude of valve-related thrombosis, while possessing longevity such that there is less need for replacement of same over the lifetime of the recipient and provides a means for ease or replacement and reduced complications associated with same.

In some aspects the valve devices of this invention specifically regulate the flow through the device. In some aspects, such regulation includes particular geometries, that in some aspects reduce turbulent flow and/or promote laminar flow through a main orifice of the device, and in some aspects, regulate non-laminar or turbulent flow through other areas of the valve device, for example, on the valve device periphery or hinge regions.

In some aspects, the valve devices of this invention provide for convex or concave positioning of the valve leaflets, which, in some aspects may be fully rotated to alter the positioning from convex to concave and vice versa.

In some aspects, the valve device may comprise ratcheting locking mechanisms around the outer rim of the device, to promote fast and secure positioning and locking of same.

### BRIEF DESCRIPTION OF THE DRAWINGS

FIGS. 1A-1F schematically depict an embodied two leaflet valve device of this invention. Referring to FIGS. 1A and 1B, the composite valve device 1-10 of this invention is depicted in posterior view. Two composite leaflets are depicted in this aspect (1-20 and 1-30); as is the valve constraining ring 1-40. Shown are internally located leaflet attachment regions 1-60 to the constraining ring, as are the internally located extending projections 1-50. FIG. 1B schematically depicts the embodied composite valve device depicted in FIG. 1A, where the leaflets have been fully opened/extended, to promote maximal blood through the voids (1-70, 1-80, 1-90) created by the positioning of the leaflets (1-20, 1-30). Internally located extending projections 1-50 are depicted as well in this embodied aspect. FIG. 1C schematically depicts the embodied composite valve device depicted in FIG. 1A, in another posterior view, where the posterior surface 1-100 is seen, more clearly depicting the concave shape formed by the leaflets (1-20, 1-30).

FIGS. 1D-1F further depict the valve as depicted in the closed position. FIG. 1D-1E schematically depict the embodied composite valve device depicted in FIG. 1A, in anterior view, where the anterior surface 1-110 is seen and both leaflets are fully in the closed position. FIG. 1F schematically depicts an embodied composite valve device in posterior view, in which the leaflets comprise further structural modifications 1-27, 1-50, which promote control of the blood flow therethrough.

FIG. 2A-2F schematically depicts an embodied tri-leaflet valve device 2-10 of this invention. Three leaflets are depicted in this aspect (2-20, 2-25 and 2-30); as is the valve constraining ring 2-40. A magnet is located within the leaflet 2-375 having matched polarity to a magnet located in the constraining ring 2-376.

FIGS. 2B and 2C schematically depicts additional aspects of the embodied valve device depicted in FIG. 2A, where the leaflets have been fully opened/extended, to promote maximal blood through a central void 2-80 created by the positioning of the leaflets and approximation of the leaflet magnet 2-374 and ring magnet 2-373. Shown are internally located extending projections 2-700 of the leaflets which participate in flow modulation.

FIGS. 2D-2F depict anterior and posterior views, respectively of an embodied tri-leaflet valve in which the leaflets are positioned in the closed position. In some aspects, the leaflet will comprise protrusions 4-700, and depressed smoother regions 4-750, which facilitates regulation of flow therethrough and I some aspects, the protrusions may vary in depth along an axis 2-372, as depicted in FIG. 2D.

FIG. 3A-3F schematically depict embodied valves of this invention. FIG. 3A schematically depicts an embodied valve assembly, wherein an implantable locking ring which can be immobilized in situ through, e.g. stitching via adherence sites 3-225 is shown. The figures further depict illustrative embodied aspects of the embodied valves as to how magnets may be incorporated in the devices of the invention and/or additional securing methods may be prepared to secure the valve in place in situ. In the figures, the accommodated fit of the magnet securing portions of the leaflets, whereby a magnet is housed in the central hollow 3-385, in both the leaflet 3-374 and the housing 3-385 and a resin/glue is applied so that it permeates through the housing recesses and the voids 3-371, which when assembled and solidified creates lateral pins 3-390 spanning across the magnet housing 3-385 into the leaflet body, creating a strong and seamless leaflet containing the magnet strongly secured therein. Similar accommodation is made for the magnets located in the constraining ring 3-373. FIG. 3F provides an exploded view of embodied elements of embodied valves of this invention with the magnets 7-375, 7-376 being incorporated in the leaflets 7-380 housing and cover 7-374 housing, or constraining ring housing and covered with the constraining ring cover 7-373, respectively. A securing means 3-225 of the constraining ring 3-40 to underlying tissue, or to additional supports used to secure the valve in situ is shown.

FIGS. 4A-4E depict other embodied elements of the valve devices of this invention, including an internal drainage port 4-400, which vents any fluid accumulating therein to a region external to the valve, for example, via a further egress channel 4-410 so that blood does not pool within internal regions of the valve devices. Also depicted are securing means 4-420, which secure embodied components of the constraining ring, which may in some aspects thread into a cognate housing in the constraining ring. In some aspects, the constraining ring is comprised of a lower ring, 4-510, which seamlessly connects to an upper ring 4-520, via securing means 4-420 attaching to cognate securing housings 4-430 in at least one element of the ring, whereby the hinge regions 4-500 of the leaflets 4-20 are simultaneously well and seamlessly secured in the constraining ring, including in some embodiments, via a securable cover 4-440 which specifically attaches over the hinge region.

FIGS. 5A-5B depict arrangement of a blood flow modulating ring device 9-600 as herein depicted, whereby the ring is placed preceding or following the valve with respect to the flow path of blood through the valve device.

FIGS. 6A-6C are photographs of embodied valve devices prepared and obtained by methods as described herein.

### DETAILED DESCRIPTION OF THE INVENTION

This invention provides a valve device, which valve comprises leaflets and a valve constraining ring, containing a first material which reduces electrostatic forces between blood and device components, reduces adhesion of blood components to said valve device or a combination thereof; and optionally a second material, which reduces electrostatic forces between blood and device components, which reduces adhesion of blood components to said valve device or a combination thereof.

According to one aspect, when a first and second material are incorporated in the valve devices of this invention, such valve devices will in turn constitute a composite valve device, as referred to herein.

According to this aspect, and representing certain embodiments of the invention, the valve device is so termed as a “composite” since the leaflets and/or constraining ring will comprise at least certain components being of the first material and certain components being of the second material.

In other aspects, and representing certain embodiments of the invention, the valve device is essentially comprised of a uniform material, which reduces electrostatic forces between the blood and device interface, reduces adhesion of blood components to said valve and/or ring device or a combination thereof.

In some embodiments, the valve devices of this invention will comprise internally located attachment regions for the leaflets, and one or more internally located extending projections, which promote regulated flow through the device.

In some embodiments, the valve devices of this invention will comprise one or more extending projections, which reduce turbulent flow through the device, or proximally thereto, which are located on the leaflets of the valve device and/or on the constraining ring component of the valve devices of this invention.

In some embodiments, this invention provides a valve device as described herein and in accordance with any embodiment and/or combination of embodiments described herein.

In some embodiments, reference to the “valve device” herein will be understood to also refer to modular device systems as herein described.

As used in the specification and claims, the singular form “a”, “an” and “the” include both singular and plural references unless the context clearly dictates otherwise. For example, the term “a leaflet” may include, and is contemplated to include, a plurality of leaflets. At times, the claims and disclosure may include terms such as “a plurality,” “one or more,” or “at least one;” however, the absence of such terms is not intended to mean, and should not be interpreted to mean, that a plurality is not conceived.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a device or method consisting essentially of the elements as defined herein would not exclude other materials or steps that do not materially affect the basic and novel characteristic(s) of the claimed invention. “Consisting of” shall mean that the devices, systems, and methods include the recited elements and exclude anything more than a trivial or inconsequential element or step. Embodiments defined by each of these transitional terms are within the scope of this disclosure.

One aspect of the present invention provides a valve device shaped and sized for attachment within an appropriate region of the heart to replace an existing heart valve. As is appreciated, reference to the valve devices of this invention is for use in heart valve replacement and/or repair. The term “valve” shall therefore be understood to encompass any appropriate structure for a heart valve, such as a mitral valve, a tricuspid valve, an aortic valve, or a pulmonary valve. While many of the examples disclosed herein are described with reference to a specific valve, it will be understood that many of the examples are not so limited and that the concepts described apply equally to other heart valves unless expressly limited herein.

The valve devices and/or valve device systems of this invention will comprise a plurality of leaflets, which are modified in structure so as to reduce electrostatic forces between blood and device components, reduce adhesion of blood components to the plurality of leaflets or a combination thereof. In some embodiments, the leaflets are comprised of a material and/or possess a geometry and/or are assembled in the device in a manner promoting proper opening and closing of the valve device, whereby regulated blood flow is facilitated therethrough, while maintaining the ability of the valve structure, as a whole, to reduce or eliminate, electrostatic forces between blood and device components, reduce adhesion of blood components to the valve components, or a combination thereof.

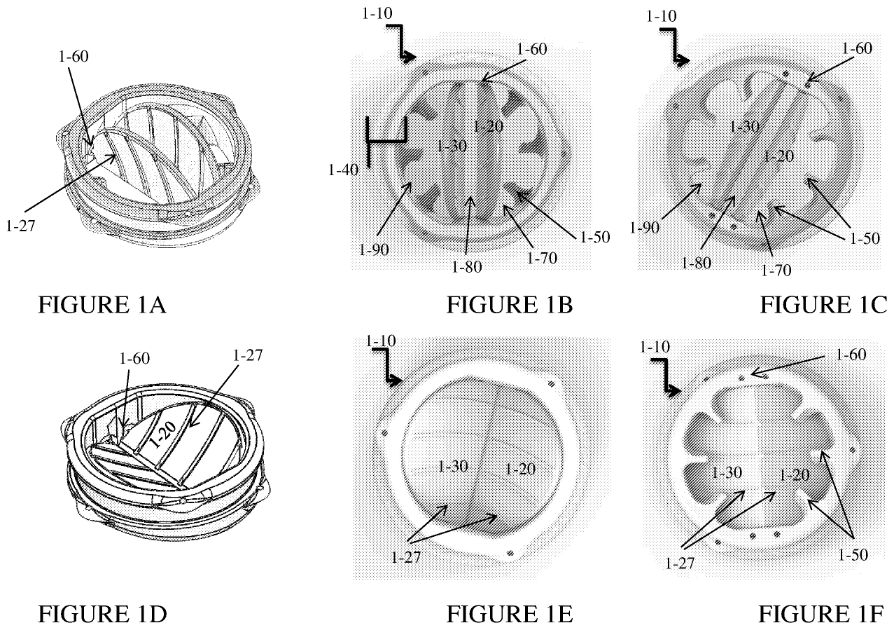

FIGS. 1-4 and FIG. 6 depict exemplified illustrative embodiments of leaflet components of the valve systems of this invention, however, the skilled artisan will appreciate that the valve systems of this invention may comprise any number or arrangement of leaflets that promote opening and closure of the valve devices while facilitating regulated blood flow therethrough with the stated advantages reducing electrostatic forces between blood and device components, reducing adhesion of blood components to the valve components or a combination thereof.

The leaflets of the valve systems as herein described will be housed within a valve constraining ring, which promotes both the opening and closure of the valve leaflets and provides a physical structure that can be appropriately affixed within the desired location to replace or support an ailing, removed or damaged cardiac valve.

In some embodiments, the valve constraining ring will be comprised of a material that reduces electrostatic forces between blood and device components, reduces adhesion of blood components to the ring or a combination thereof.

In some embodiments, the valve constraining ring will possess structural modifications that reduces electrostatic forces between blood and device components, reduces adhesion of blood components to the ring or a combination thereof. In some embodiments, the structural modifications to the valve constraining ring include projections extending from the outer ring framework toward the ring center, which in some embodiments, are referred to as extending projections, which in some embodiments, extend in a periodic manner from the ring boundary toward the ring center, or in some embodiments, extend in an aperiodic manner from the ring boundary toward the ring center, In some embodiments, one or more extensions are present in the constraining ring, and in some embodiments, the structure of each extension is substantially similar in length, angle and/or curvature, and in some embodiments, the structure of each extension varies in terms of length, angle and/or curvature.

The valve devices and/or valve device systems of this invention will comprise two or more leaflets, a valve constraining ring or a combination thereof, which in some embodiments are comprised of a first and second material as herein described in any orientation or arrangement, thereby representing a composite structure.

The term “composite” refers to a structure that may incorporate more than one material therein. The valve and/or ring devices of this invention, in some embodiments, are composite structures, since one or more components may be comprised of different materials.

In some aspects, the valve and/or ring device may resemble a “composite structure” when the various components or elements, even if comprised of identical materials, will have a different structure, thickness, finishing, or other discrete differences, so that desired structural characteristics will be obtained, although there is a single chemical entity of which the device is comprised.

It is to be understood therefore that the devices and/or rings and/or assemblies of this invention contemplate use of a single material for preparing the components of the device, although, various embodiments of the invention indeed refer to the use of different materials for different components of the devices and assemblies described herein.

The valve devices and assemblies of this invention will comprise leaflets. In some aspects, the leaflet is a composite structure and in some embodiments, the leaflet is comprised of a single material, which reduces electrostatic forces between blood and device components, reduces adhesion of blood components to said valve and/or ring device or a combination thereof.

The term “leaflet” in some embodiments, refers to cusps, or structures of the valve that seam together and separate, as part of the valve opening and closing.

Referring to FIGS. 1A-1F, elements 1-20 and 1-30 are depicted, which are embodied schematic examples of leaflets. According to this aspect and in some embodiments, the valve systems of this invention will contain 2 leaflets, and in other embodiments, the valve systems of this invention will contain 3 leaflets, or, in other embodiments, the valve systems of this invention will contain 4 leaflets. It will be appreciated that the valve systems of this invention will not be limited in terms of the number of leaflets contained therein.

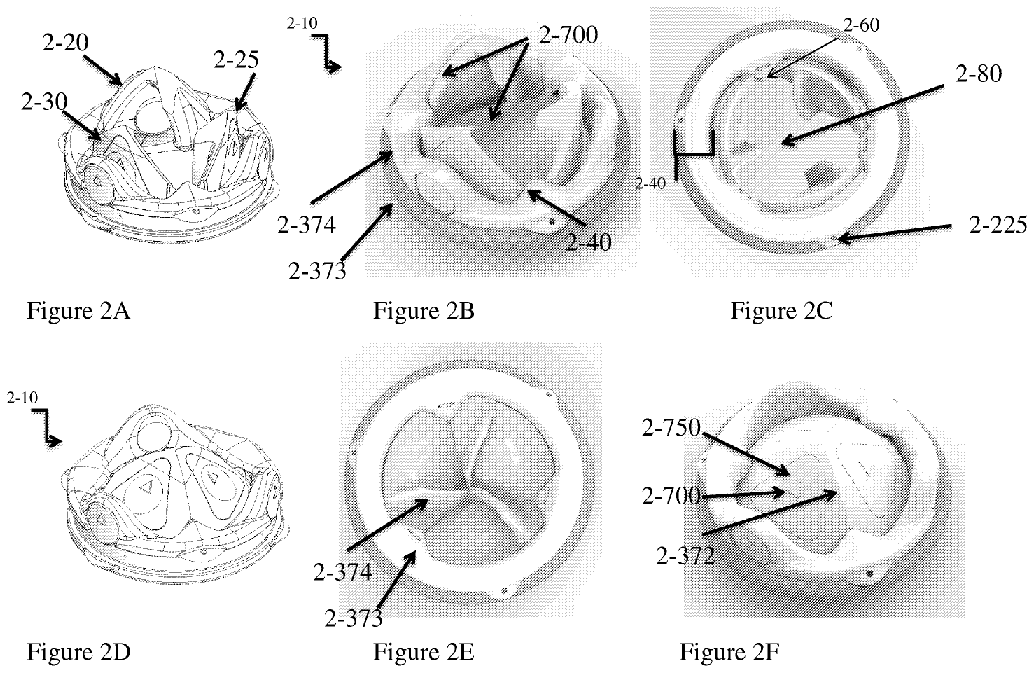

In some aspects the leaflet surface will be as smooth as possible and in some embodiments, the leaflet may be contoured to promote blood flow through an opening created by the rotation of the leaflet about an axis, which in some embodiments, creates a main conduit in the center of the valve system when the leaflets are in the open position. For example, referring to the embodied valve leaflets as depicted in FIGS. 2A, 2B and 2E, the leaflet may be contoured to promote blood flow through an opening created by the rotation of the leaflet about an axis, which in some embodiments, creates a smaller conduit in the center of the valve system 2-80, when the leaflets are in the open position, for example as depicted in FIGS. 2B and 2C.

In some aspects, the modification of the leaflets serves to provide additional mechanical strength to the leaflets, such as, for example, the ridged 1-27 modifications to the leaflets as depicted in FIGS. 1E-1F, and such modifications may be present on an anterior or posterior surface of the leaflet, or both.

In some aspects, the structural modifications of the leaflets create a protrusion from the surface of the leaflet, which in some embodiments, may be at any desired angle therefrom, such as, for example, from 1 to 90 degrees, and in some embodiments, the protrusion from the surface of the leaflet may comprise regions that vary in terms of the angle as described such that regions closer to the leaflet region joining another leaflet have varying heights and angles as compared to regions closer to the leaflet attachment region to the constraining ring.

In some embodiments, the leaflet may be anchored at its base at a desired position in the constraining ring so that rotation about the anchor point pivots the apical portion of the leaflets toward the center to enable closure. In other embodiments, the leaflet may be anchored at its base at a desired position in the constraining ring so that rotation about the anchor point pivots the apical portion of the leaflets laterally to the closed position.

It will be understood that the leaflets may also be anchored in a central position in the valve or be anchored in more than one position so that rotation may be 360 degrees about a desired axis for opening or closing, as will be understood by the skilled artisan, and the invention contemplates other arrangements of the leaflets of the valve systems of this invention.

In some embodiments, the leaflet of the valve devices of this invention and/or the constraining rings and/or other components of any of the devices of this invention are comprised of at least a first material which reduces electrostatic forces between blood components, or at least a first material which reduces adhesion of blood components to a valve device of this invention or a combination thereof.

According to this aspect, and in some embodiments, such material, may include a ceramic material, or a layer or block of ceramic material.

In some embodiments, the material, which reduces electrostatic forces between blood and valve device components, or reduces adhesion of blood components to a valve device of this invention may include a Teflon material or a layer of Teflon material, and the like.

In some embodiments, the leaflet of the valve devices of this invention and/or the constraining rings and/or other components of any of the devices of this invention are comprised of at least a second material, which reduces electrostatic forces between blood components, or reduces adhesion of blood components to a valve device of this invention, and the valve and ring devices are thereby composite devices, as herein described.

According to this aspect and in one embodiment, the leaflet of the valve devices of this invention and/or the constraining rings and/or other components of any of the devices of this invention are comprised of a second material, which similarly reduces electrostatic forces between blood components, or reduces adhesion of blood components to a valve device of this invention, and the valve and ring devices are thereby composite devices, as herein described, but is a different material than the first material, or in some embodiments, is the same material but varied in terms of thickness, or other structural characteristics regarding same.

In other embodiments, the second material may possess different properties from the first material, and may, for example, be present in minute quantities.

In some embodiments, the valve devices of this invention will be comprised of silicon carbide, Zirconia toughened alumina (ZTA), silicon nitride, boron carbide, nanocrystalline zirconium oxide and other nanoceramics or combinations thereof. [
In some embodiments, the valve devices of this invention will comprise a yittria stabilized zirconium-toughened alumina ceramic product.
In some embodiments, methods of preparing ceramic products as appearing in U.S. Pat. Nos. 6,624,106, 8,012,897 and 7,402,541 will be modified to produce the valve devices as herein described, and the broad description of the materials and means for preparing same as appearing in the stated patents are fully incorporated herein by reference. It will be appreciated that the skilled artisan will know how to modify the teachings of these references to scale down the ceramic products and to finish same to be suitable for the instsant invention.

In other embodiments, the material may comprise aluminum oxide, optionally in combination with a composite material as herein described.

In some embodiments, the valve and/or ring devices of this invention will be comprised of any biocompatible ceramic material.

In some embodiments, the first material and second material are located on surface exposed regions of at least one leaflet. In some embodiments, the first material and second material are located on the same surface exposed regions of the leaflet or in any given section of the valve structures of this invention, whereby the first material is a more apically located layer and second material is a more basally located layer. In other embodiments, the first material and second material are located on the same surface exposed regions of the leaflet or in any given section of the valve of this invention, whereby the first material is a more basally located layer and the second material is a more apically located layer.

In some embodiments, the first material and second material are located on different surface exposed regions of the leaflet or in any given section of the valve structures of this invention.

In some embodiments, the device comprises two or more leaflets and the two or more leaflets comprise a first material on a first surface of the leaflet and second material on an opposing surface of the leaflet. In some embodiments, the device comprises two or more leaflets and a first leaflet comprises the first material on a first surface of the leaflet and a second of the leaflets comprises a second material on a first surface of the leaflet. In some embodiments, a first leaflet of two or more leaflets comprises a second material on a second surface of the leaflet and a second leaflet comprises a first material on a second surface of the leaflet.

In some embodiments, the leaflet will comprise a specialized structure on its surface, which comes into contact with blood flow proximally thereto.

According to this aspect, and in some embodiments, such a specialized structure may comprise at least a first region that protrudes and in some embodiments, such specialized structure may further comprise at least a second region, which is recessed.

According to this aspect, and in some embodiments, such protruding region may be angled and/or rounded and/or ridged and/or contoured to ensure a smooth surface in contact with the blood and its components in contact therewith.

In some embodiments, such second region, which is recessed may be angled and/or rounded and/or contoured to ensure a smooth surface in contact with the blood and its components in contact therewith.

According to this aspect, and in some embodiments, such a specialized structure will promote laminar flow or reduce turbulent flow, or otherwise facilitate reduction in the potential for thrombogenesis in situ.

In some embodiments, such a specialized structure extends in a smooth manner perpendicularly with respect to the long axis along the width of the leaflet, and such extension region in some embodiments is located off center with respect to the perpendicular axis of the leaflet.

In some embodiments, the length of the specialized structure approximates the height axis of the leaflet. In some embodiments, the length of the specialized structure is from 30-95% of the height axis of the leaflet. In some embodiments, the length of the specialized structure is from 10-30% of the height axis of the leaflet.

In some embodiments the width of the specialized structure may be of any appropriate width to create the desired contour.

In some aspects, such specialized structure will promote positioning such that flow is strongest and least impacted at the center of the passageway created in the valve devices of this invention. In some aspects, such specialized structure will promote positioning such that flow is strongest and least impacted at the center of the main passageways created in the valve devices of this invention, which in some aspects is not at the center of the valve device.

The devices of this invention are so constructed such that under flow conditions, the leaflets become separated and move about the securing region axis, to an open position.

In some embodiments the overall shape and three dimensional structure of the leaflet is constructed so as to reduce turbulent flow, and in some embodiments, to promote laminar flow. In some embodiments the leaflets are contoured such that an inner surface is concave, or, in some embodiments, at least slightly concave, or in some embodiments, at least substantially concave.

According to this aspect and in other embodiments, the leaflets are further constructed such that an outer surface is convex, or, in some embodiments, at least slightly convex, or in some embodiments, at least substantially convex.

It will be appreciated that regardless of the contouring of the leaflets, the leaflets of the devices of this invention meet and form a sufficiently tight junction such that backflow through the valve device is substantially prevented.

In some embodiments, a marked advantage of the valve systems of the instant invention is the ability to diminish or reduce backflow through the valve systems of this invention.

In some aspects, the leaflet will comprise protrusions, and depressed smoother regions. In some aspects, the protrusions are angled outward in an angle of from 0 to 90 degrees perpendicular to the axis of the leaflet. In some aspects, the protrusions are rounded and in some embodiments, at least an edge of the protrusion is angled and in some embodiments at least an edge of the protrusion is perpendicular to the long axis of the leaflet.

Referring to FIGS. 1B, 1C, 1E, 1F, 2B, 2C and 2E, certain illustrative non-limiting examples of protrusions (elements 27, 374 and 700) are depicted, as are the depressed smoother regions (element 250 and 750). It will be appreciated that any number of additional shapes, lengths, angles, etc. of the protrusions are envisioned, and that same may be scaled or otherwise modified to suit an array of mechanical devices, which may further comprise any embodiment as described herein.

In some embodiments, the leaflet will comprise at least a first small magnet incorporated within said leaflet structure having a first polarity.

Such first magnet may comprise any appropriate material, such as, for example, Neodymium coated with a biocompatible polymer, such as silicon nitride, silicon carbide, titanium nitride and others, as will be appreciated by the skilled artisan. In some aspects, the first magnet may comprise pyrolytic carbon (PyC).

In some aspects, the inclusion of the magnets promotes ease of closure of the valve, which, in some embodiments, is immediate and in other embodiments, prevents blood backflow therethrough.

In some aspects, when the leaflets and constraining ring contain proximally located magnets of matched polarity, repulsive forces between the two magnets exist and when the magnets within the leaflet and magnets within the valve constraining ring are brought into proximity, repulsion occurs, which is overcome under flow conditions, maintaining the valve in the open position. In the absence of flow through, the repulsive forces are sufficient such that the leaflets are propelled away from the constraining ring, to resume a closed position.

In some embodiments, one or more additional magnets may be incorporated in each leaflet, or in different leaflets, including, incorporation of weaker magnets of opposing polarity to the first magnet in the leaflet. According to this aspect, such incorporation of a weaker magnet of opposing polarity may promote a faster closure.

In other embodiments, the leaflets may comprise a terminal extension, which in turn acts as a stopper, or in some aspects such extension slows closure of the valve. In some embodiments, the leaflets may comprise a terminal extension, which in turn acts as a stopper, or in some aspects such extension hastens the closure of the valve.

The valve devices of this invention further comprise a valve constraining ring.

The valve constraining ring will be constituted, in some aspects, of at least a first material which reduces electrostatic forces between blood and valve device components; or reduces adhesion of blood and device components or a combination thereof and, in some embodiments, the valve constraining ring will optionally be constituted, in some aspects, of at least a second material, which may reduce electrostatic forces between blood and valve device components; or reduces adhesion of of blood and device components or a combination thereof, in some embodiments. In some embodiments, the second material imparts strength to the overall device structure.

In some embodiments, the valve constraining ring will comprise internally located attachment regions for the leaflets of the valve as described herein.

In some embodiments, the internal attachment regions may further comprise a drainage port, which vents any fluid accumulating therein to a region external to the valve. In some embodiments the valve constraining ring may further comprise an egress channel so that blood does not pool within internal regions of the valve devices.

In some embodiments, the valve constraining ring will optionally comprise at least a second magnet of matched polarity to the first magnet located on one or more leaflets of the valve devices as described herein. According to this aspect, and in some embodiments, the at least a second magnet of matched polarity will be incorporated within the valve constraining ring at regions proximal to the first magnet within one or more leaflets such that repulsive forces are present when the first magnet and second magnet are in proximity with each other in the absence of flow.

In some embodiments, the valve constraining ring will comprise at least one extending projection internally located within said ring, which promotes regulated flow therethrough.

Referring to FIGS. 1B, 1C and 1F, certain illustrative examples are schematically depicted at element 50, where extending projections internally located within the ring are shown, which projections promote regulated flow therethrough.

In some embodiments, the term “regulated flow” refers to an influence on blood flow through a valve device of this invention, or in some embodiments, the term “regulated flow” refers to an influence on blood flow proximal to a valve device of this invention, which reduces the turbulence of the flow, or in some embodiments, promotes linear or laminar flow, in other embodiments. In some embodiments, the term “regulated flow” provides a flow that reduces the likelihood of thrombogenesis at or near the implanted valve device.

In some embodiments, the closing of the leaflet is facilitated by the shape of the leaflet, for example, in some embodiments, by elongating the end of the leaflet, to promote closure of same in the absence of a strong flow, so that the magnetic repulsion is not required for closure of the leaflets.

In other embodiments, the closing of the leaflet is facilitated by the shape of the leaflet, for example, in some embodiments, by increasing the thickness or otherwise creating heavier regions in the leaflet, to promote rotation of same about an axis at the juncture between the leaflet and constraining ring, to promote closure of same in the absence of a strong flow, so that the magnetic repulsion is not required for closure of the leaflets.

It will be appreciated that the valve constraining ring will comprise at least one extending projection internally located within the ring, which projection is not to be limited in terms of angle, periodicity, size, geometry, etc. as multiple examples have been shown for illustration and the skilled artisan will readily appreciate that an extending projection projecting into the lumen of the ring will participate and impact reduction of turbulent flow therethrough.

In some embodiments, the number of leaflets is a function of the envisioned ideal flow and fit through same for a particular heart, and in some embodiments, the choice of same is not necessarily reflective of a similar structure to the native valve being replaced.

In some embodiments, the specialized structures in the leaflets combined with the specialized structures of the constraining ring together promote facilitating still greater laminar flow, or in some embodiments, further reduced turbulent flow, or in some embodiments, otherwise further facilitating reduction in the potential for thrombogenesis in situ, including any combination of advantages as herein described.

In some embodiments, the valve constraining ring further comprises an externally located region that can be affixed appropriately to underlying cardiac tissue or to proximally located blood vessels to secure the valve in place, as needed.

In some aspects, the valve constraining ring may further comprise at least one externally located ratcheting locking mechanism. According to this aspect, and in one embodiment, such ratcheting locking mechanism accommodates the insertion of a suture-like structure therethrough, whose advancement is uni-directional and regulated.

The valve constraining ring of this invention may also be a composite of two materials. In some aspects, the valve constraining ring is comprised of at least a first material which reduces electrostatic forces between blood and device components, reduces adhesion of blood components to device components or a combination thereof. Such material will comprise any material thus described hereinabove.

In some embodiments, the valve constraining ring of the composite valve devices of this invention are comprised of at least a second material, which in some embodiments reduces electrostatic forces between blood and device components, reduces adhesion of blood components to the device components or a combination thereof. In other embodiments, the second material may be comprised of any material or embodiment as herein described.

In some embodiments, the first material and the second material are the same material, with respect to any of the valve and/or constraining ring or components thereof.

In some embodiments, the leaflets, valve constraining rings or combination thereof may be a composite of the first and second materials as described hereinabove.

In some embodiments, the first material and second material are located on surface exposed regions of the valve constraining ring, in a side-by-side manner. In some embodiments, the first material and second material are located on the same surface exposed regions of the valve constraining ring, whereby the first material is a more apically located layer and second material is a more basally located layer. In other embodiments, the first material and second material are located on the same surface exposed regions of the valve constraining ring, whereby the first material is a more basally located layer and the second material is a more apically located layer.

In some embodiments, the first material and second material are located on different surface exposed regions of the valve constraining ring, for example, in some embodiments, the leaflet(s) are comprised of the first material and the constraining ring is comprised of the second material, or vice versa.

It will be appreciated that the orientation and placement of the first and second material may vary whether in the leaflet(s), valve constraining ring or combination thereof, and any such variation in placement is to be considered as part of this invention.

It will also be appreciated that the valve devices of this invention may comprise two or more leaflets, which two or more leaflets may in turn comprise two or more first materials, second materials or combinations of same for example, a first leaflet may comprise a first ceramic material and a second leaflet may comprise a second ceramic material and the valve constraining ring may comprise the first, or second ceramic material or a third ceramic material or combinations thereof. Similarly, the second material may be varied with respect to the leaflets, valve constraining rings or combinations thereof and all are to be considered as part of the invention.

In other aspects, the at least one composite leaflet, valve constraining ring, or a combination thereof is comprised of at least a first region and a second region that possess comparable structural characteristics, for example, in terms of hardness or strength.

In another embodiment of the invention, the valve device, valve constraining ring is made from bio-compatible materials.

In some embodiments, the valve device is comprised of a metal, a polymeric material, or a combination or composite thereof. In some embodiments, the valve device is comprised of nitinol, stainless steel, PTFE, polyester, silicon or a combination or composite thereof. According to this aspect, the surface exposed layer will be comprised of a biocompatible ceramic material, as herein described and this layer is what is in contact with blood and tissue of the subject into which the valve devices of this invention are implanted.

In some embodiments, this invention provides a valve device, comprising:
two or more leaflets; comprising:
   at least a first material which reduces electrostatic forces between blood components and device components, reduces adhesion of blood components to said two or more leaflets or a combination thereof; and
   optionally at least a second material, which reduces electrostatic forces between blood components and device components, reduces adhesion of blood components to said two or more leaflets or a combination thereof; and
   optionally at least a first magnet incorporated within said leaflet structure; and
a valve constraining ring, comprising:
   at least a first material which reduces electrostatic forces between blood components and device components, reduces adhesion of blood components to said valve constraining ring or a combination thereof; and
   optionally at least a second material, which reduces electrostatic forces between blood components and device components, reduces adhesion of blood components to said valve constraining ring or a combination thereof; and
   internally located attachment regions for said two or more leaflets; and
   optionally at least a second magnet of matched polarity to said first magnet, incorporated within the valve constraining ring at regions proximal to said first magnet within said leaflet such that repulsive forces are present when said first magnet and said second magnet are brought into proximity with each other in the absence of flow.

In some embodiments, the valve device is comprised of a base biocompatible material, as described herein and may further comprise a coating.

In another embodiment of the invention, the valve constraining ring will comprise various securing means allowing for the securing of the composite valve device to proximally located cardiac tissue or proximately located blood vessels. The securing means may include any appropriate known means, such as sutures, adhesives, staples, and mechanical fasteners, and the ratcheting locking mechanism as herein described.

The suturing guide structures are appropriate for any envisioned suture, from any known material and introduced via any appropriate technique such as, for example, everted mattress sutures, non-everting mattress sutures, figure of eight sutures or continuous sutures. Other attachment means are envisioned for use, as well.

This invention therefore also provides a kit of parts, which comprises one or more valves as herein described, whereby the choice of valve may be selected in real time, as part of an optimized implantation procedure.

In some aspects, such kits may further comprise various sutures and other fasteners, as well, including any appropriate fastener or suture as known in the art or as described herein.

This invention also provides a modular valve device, wherein the valve systems as herein described can be secured in situ within a permanently implanted housing, facilitating easier valve replacement in repeat procedures.

This invention provides, in some embodiments, a method of performing cardiac valve repair or replacement surgery on a patient comprising the steps of applying the valve device as described herein, in accordance with any embodiment as described herein to replace a defective or malfunctioning valve in a subject.

In some embodiments, the methods of this invention specifically relate to performing repeat cardiac valve repair or replacement surgery on a patient comprising the steps of replacing a reversibly lockable mechanical valve device of a modular valve assembly as herein described implanted in a subject with at least one new reversibly lockable mechanical valve device, which new reversibly lockable mechanical valve device is releasably secured within the implantable securable locking ring, following removal of the previous lockable mechanical valve device.

Referring now to the Figures, which help illustrate some aspects of the invention, FIGS. 1A-1F schematically depict non-limiting, illustrative embodied bi-leaflet valve devices 1-10 of this invention. FIG. 1A depicts the valve in the open position, and FIGS. 1B and 1C depict the same device in open position illustrating the anterior and posterior views, respectively. Two leaflets are depicted in this aspect (1-20 and 1-30); which leaflets are secured to the valve constraining ring 1-40 via the internally located attachment regions 1-60. The leaflets are so constructed such that at least partial rotation about an axis is attainable, so that a concave or convex shape is facilitated, which choice is reflective, as will be understood by the skilled artisan, of the device positioning desired.

It will be also appreciated, as depicted, for example in FIGS. 1B-1C that full rotation of the leaflets fully exposes the voids to promote maximal flow therethrough. In another aspect, internally located extending projections 1-50 are present, which projections, in turn influence the blood flow therethrough or proximal thereto. For example, and in some aspects, flow in regions most proximal to the projections may be non-linear or turbulent, whereas flow through a central void will be linear and such varied flow may in turn decrease the thrombotic events that the valve devices of this invention in some embodiments, specifically address.

As described herein the invention contemplates the leaflets comprising a first and second material as herein described. In some aspects, the first material and second material may be layers of same, with the first material being more surface exposed on both anterior and posterior aspects of the leaflets. In some aspects, the first material and second material may be layers of same, with the first material being more surface exposed on an anterior aspect of the leaflet (e.g. the exposed leaflet perspective in FIG. 1E), and the second material being more surface exposed on a posterior aspect of the leaflets (e.g. the exposed leaflet perspective in FIG. 1F). Similarly, for example, referring to FIG. 1E, the first material being more surface exposed on a first leaflet (e.g. 1-20) and the second material being more surface exposed on a second leaflet (e.g. 1-30), or vice versa. The orientation can be thus manipulated with respect to anterior or posterior surface exposed aspects of the leaflets, as well.

FIG. 1F schematically depicts am embodied composite valve device in posterior view, in which the leaflets comprise further structural modifications 1-27, which promote control of the blood flow and/or provide additional strength to the leaflet. As is depicted in this embodied illustration, ridges, which, in some embodiments, may be angled to be of a greater height at regions proximally placed to the constraining ring, may modulate the turbulence of the flow through the valve.

FIGS. 2A-2F, similar to FIGS. 1A-1F schematically depict non-limiting, illustrative, embodied valve devices 2-10 of this invention and in these figures, the device comprises three leaflets (2-20, 2-25 and 2-30); attached via internal attachment regions 2-60 to the valve constraining ring 2-40. FIG. 2B schematically depicts the embodied valve device where the leaflets have been fully opened/extended, to promote maximal blood through the central void (2-80) created by the positioning of the leaflets. As noted, the extending projections (2-700) serve to promote regulated flow, for example promoting less linear flow through lateral voids, than the flow through the central void 2-80, which would be substantially linear. Magnets have been incorporated in specialized compartments in the leaflets and constraining ring, 2-374 and 2-373, respectively, with the magnets being of similar polarity, such that under the pressure of flow, the repulsive forces are overcome but otherwise are sufficient to promote closure of the leaflets and distancing of the leaflet magnet from the constraining ring contained magnet. FIGS. 2C and 2F provide a posterior view, where leaflet protrusions 2-700 are present, which regulate flow to promote less turbulent or flow approximating laminar flow through the central void 2-80. Protruding regions 2-700 may be proximal to more recessed regions 2-750, which in some aspects promote the regulated flow. Seaming of the three leaflets 2-372 may occur by structural modification of the protrusions such that same do not interfere with proper seaming of the leaflets.

In some embodiments, this invention provides a modular valve assembly, comprising:
an implantable locking ring into which a mechanical valve device inserts, and is reversibly locked therewithin; and
a reversibly lockable mechanical valve device comprising at least one reversible locking mechanism insert, which reversible locking mechanism insert is reversibly securable within said implantable locking ring.

The term “modular valve assembly” refers, in some embodiments, to a reversibly lockable mechanical valve device, which is reversibly constrained within an implantable locking ring, such that the mechanical valve device can be locked within and alternately removed from the locking ring.

According to this aspect, when the mechanical valve device is locked within the locking ring, the valve device is secured and functional. Also according to this aspect, when it is desired to replace the mechanical valve device, same may be readily accomplished via specific release of the mechanical valve device from the locking ring. According to this aspect, the device is a modular assembly in that a component of the assembly remains implanted while an additional component is removable and replaceable.

It is to be appreciated that the modular valve assembly is a non-limiting example of an embodiment envisioned for the instant invention, and that the invention contemplates same as an optional iteration of the valve devices as herein described, but there is no requirement to the valve devices of this invention to be modular in terms of their ability to be releasably contained within a ring implanted in a subject for a longer period of time as compared to the valve implantation.

In other embodiments, the locking ring may contain the releasable locking mechanism, which inserts within a cognate appropriate structure in the mechanical valve device. It will be appreciated that the ability to engage and disengage the mechanical valve device from the locking ring fulfills the envisioned aspect of the invention to provide a modular composite valve assembly which may be releasably secured, and the invention contemplates either part of the releasable locking mechanism and cognate “catch” structure being located within the mechanical valve device and locking ring, respectively.

It will be appreciated that the embodied modular valve assemblies comprise leaflets as herein described incorporated in same, and in some aspects, including embodied valve devices comprising the leaflet and constraining ring magnets, which can readily be accommodated and releasably inserted and locked within an implantable locking ring as herein described. According to this aspect, and in some embodiments, such systems may optionally further comprise at least one relaseable locking mechanism insert, which relaseable locking mechanism insert is releasably securable within the locking ring, so that the embodied modular valve devices can readily constitute a part of the modular valve assemblies as herein described.

Another aspect of the present invention provides a modular valve device shaped and sized for attachment within an appropriate region of the heart or affected blood vessel to replace an existing valve and another aspect of the present invention provides a modular valve device, which provides for replacement of the mechanical valve device component of the assembly, within a stably inserted/implanted locking ring. As is appreciated, reference to the valve devices/assemblies of this invention is for use in heart valve replacement and/or repair.

The modular valve devices of this invention will comprise a modular mechanical valve, permanently or more permanently implanted locking ring into which such modular mechanical valve is releasably incorporated, which modular valve comprises a leaflet, a valve constraining ring or a combination thereof, according to any embodiment as herein described.

In some aspects, the valve constraining ring and/or locking ring may further comprise at least one externally located ratcheting locking mechanism. According to this aspect, and in one embodiment, such ratcheting locking mechanism accommodates the insertion of a suture-like structure therethrough, whose advancement is uni-directional and regulated.

The valve constraining ring and/or locking ring of this invention may also be a composite of two materials, such as, for example, at least a first material which reduces electrostatic forces between blood components, reduces adhesion of blood components to said leaflets or a combination thereof. Such material will comprise any material thus described hereinabove.

In some embodiments, the valve constraining ring of the composite valve devices of this invention are comprised of at least a second material, which material reduces electrostatic forces between blood and valve components, reduces adhesion of blood components to said valve components or a combination thereof and in turn may be comprised of any such material or embodiment as herein described.

In some embodiments, the leaflets, valve constraining rings, and/or locking ring or combination thereof may be a composite of the first and second materials as described herein.

In some embodiments, the first material and second material are located on surface exposed regions of the valve constraining ring, in a side-by-side manner. In some embodiments, the first material and second material are located on the same surface exposed regions of the valve constraining ring, whereby the first material is a more superficially located layer and second material is a more deep layer. In other embodiments, the first material and second material are located on the same surface exposed regions of the valve constraining ring, whereby the first material is a more basally located layer and the second material is a more apically located layer.

In some embodiments, the first material and second material are located on different surface exposed regions of the valve constraining ring.

It will be appreciated that the orientation and placement of the first and second material may vary whether in the leaflet(s), valve constraining ring or combination thereof, and any such variation in placement is to be considered as part of this invention.

In another embodiment of the invention, the locking ring will comprise various securing means allowing for the securing of the modular assemblies of this invention to proximally located cardiac tissue. The securing means may include any appropriate known means, such as sutures, adhesives, staples, and mechanical fasteners, and the ratcheting locking mechanism as herein described.

The suturing guide structures are appropriate for any appropriate envisioned suture, from any known material and introduced via any appropriate technique such as, for example, everted mattress sutures, non-everting mattress sutures, figure of eight sutures or continuous sutures. Other attachment means are envisioned for use, as well.

This invention therefore also provides a kit of parts, which comprises one or more valves as herein described, whereby the choice of valve may be selected in real time, as part of an optimized implantation procedure.

In some aspects, such kits may further comprise various sutures and other fasteners, as well, including any appropriate fastener or suture as known in the art or as described herein.

In some aspects, with respect to the mechanical valve device of the assemblies of this invention, the leaflets of the device are so constructed such that at least partial rotation about an axis is attainable, so that a concave or convex shape is facilitated, which choice is reflective, as will be understood by the skilled artisan, when mitral or aortic positioning is desired. It will be also appreciated, that full rotation of the leaflets fully exposes the voids to promote maximal flow therethrough. In another aspect, internally located extending projections are present, which projections, in turn influence the blood flow therethrough. For example, and in some aspects, flow in regions most proximal to the projections may be non-linear or turbulent, whereas flow through a central void will be linear and such varied flow may in turn decrease the thrombotic events that the valve devices of this invention in some embodiments, specifically address.

This invention provides for the use of the valve assemblies of this invention in cardiac valve repair or replacement surgery in a patient in need of same, where the implanted assembly replaces a defective or malfunctioning valve in a subject.

This invention provides further for the use of the modular valve assemblies of this invention in repeat cardiac valve repair or replacement surgery in a patient in need of same. For example, and in some embodiments, the mechanical valve device may be less operable over time, or less efficiently operable, or demonstrate tissue debris buildup, or may be otherwise improved upon by newer mechanical valve device models, such that it would be desirable to replace specifically the mechanical valve component of the assembly of the invention.

According to this aspect and in some embodiments, uniquely, the mechanical valve device may be specifically replaced from the modular valve assemblies, as herein described, with at least one new reversibly lockable mechanical valve device, which new reversibly lockable mechanical valve device is releasably secured within said implantable securable locking ring, following removal of the previous lockable mechanical valve device.

In some aspects, such replacement may be accomplished in a minimally invasive and safer procedure, for example, than insertion of a new mechanical device via conventional means.

This invention provides for the use of the valve devices of this invention to replace or promote repair by replacement of a faulty or diseased valve in a subject in need thereof. Thus, in some embodiments, this invention provides a method of performing cardiac valve repair or replacement surgery on a patient comprising the steps of applying the valve device of this invention to replace a defective or malfunctioning valve in a subject

In some aspects, the invention provides for reduced need to administer anti-coagulation therapy to a subject provided with a valve of this invention including any embodiment for same as herein described.

In some embodiments, this invention provides a valve device, comprising two or more leaflets, as described herein and according to any embodiment as herein described, optionally further comprising at least a first magnet incorporated within at least one leaflet structure in the valve, and the invention further comprises a valve constraining ring, as described herein and according to any embodiment as herein described, further comprising at least a second magnet of matched polarity to that of the first magnet, incorporated within the valve constraining ring at regions proximal to the first magnet such that repulsive forces are present when the first magnet and second magnet are brought into proximity with each other in the absence of flow. According to this aspect and in some embodiments, the valve devices and/or modular valve assemblies of this invention will include the first and second magnets as herein described.

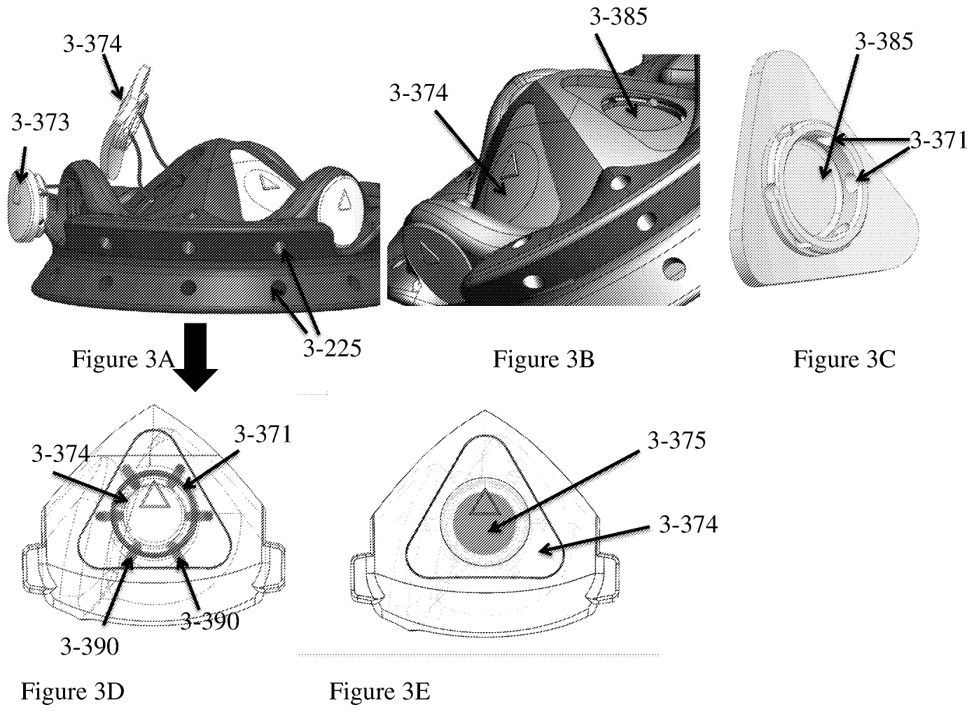

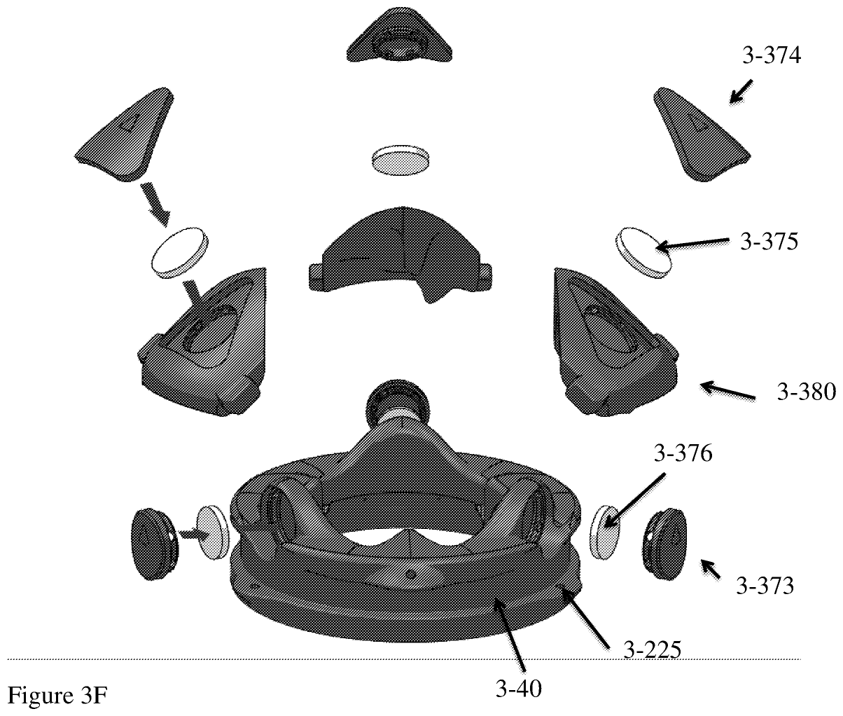

FIG. 3A-3E schematically depict still further embodied valve devices and aspects of same. The valve can be secured in situ via suturing through the attachment regions depicted 3-225. While FIG. 3A-3E depicts embodied devices comprising first and second magnets, it will be readily appreciated that similarly structured, bi-, tri-leaflet valves without magnets positioned on the leaflets are envisioned, wherein the leaflet design and extending proximal regions of the constraining ring, which come into contact with the leaflets when same are in the opened position, when exposed to flow, serve to limit the rotation of the leaflet and promote their closure, as well.

For example, as will be appreciated by the skilled artisan, the apical regions of the leaflets may be contoured, weighted, seamed, or contain any structural modification that would promote a more likely closure, to which a force equal to that of blood flow would overcome in order to promote leaflet separation and opening.

FIGS. 3A-3F depict valve devices, which may be prepared by any appropriate means, including three-dimensional printing of same, whereby the magnets are contained in a seamless housing as described further in the Examples below.

In some embodiments, and referring to FIGS. 3A and 3D additional means for enclosing the magnets within the leaflets and securing the magnets within proximal regions of the constraining ring are illustrated.

According to this aspect and in some embodiments, it will be appreciated that it is useful and in some aspects required to secure the magnets within a seamless framework in the leaflets and ring as herein described. As herein described, magnet covers 3-374 on the leaflets and magnet covers on the securing ring 3-373 are held in place, for example, by being glued with an appropriate epoxy resin or other biocompatible resin and adhered to the cognate recess/compartment in the leaflet and ring housing.

Referring to FIGS. 3B and 3C, a series of recessed holes 3-371 are created in the recessed magnet housing 3-385 in the leaflet. Similarly, a series of recessed holes 3-371 are created in the recessed magnet housing 3-385 in the leaflet cover 3-374. When the magnet is inserted and the adhesive is applied, the adhesive coats and infiltrates the entire housing surface and extends laterally into the indicated recessed holes such that upon polymerization/hardening the adhesive solidifies and forms a strong pin-inserting 3-390 locking mechanism to firmly secure the magnet within the seamless leaflet housing. FIG. 3F provides an exploded view which facilitates appreciation for the assembly of the valve device components.

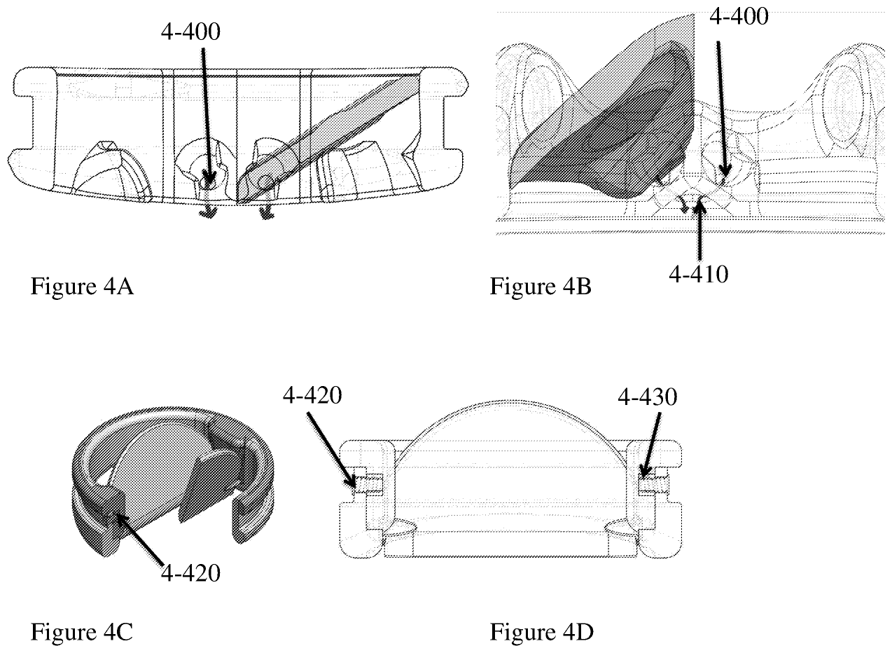

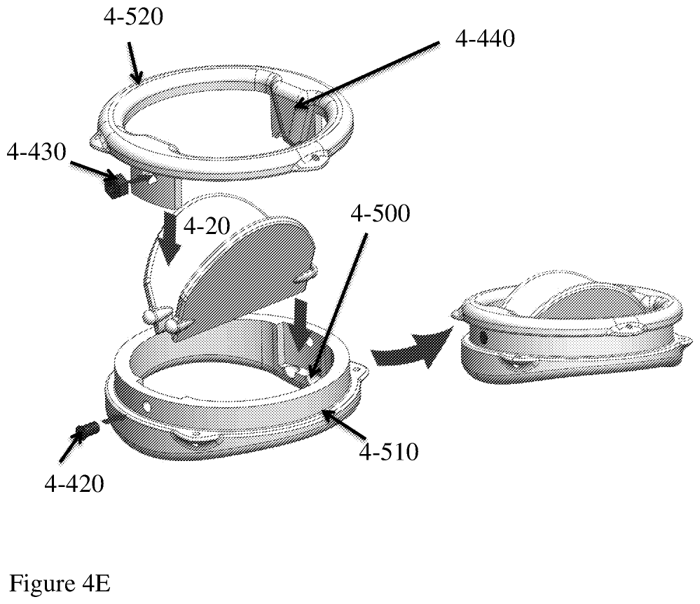

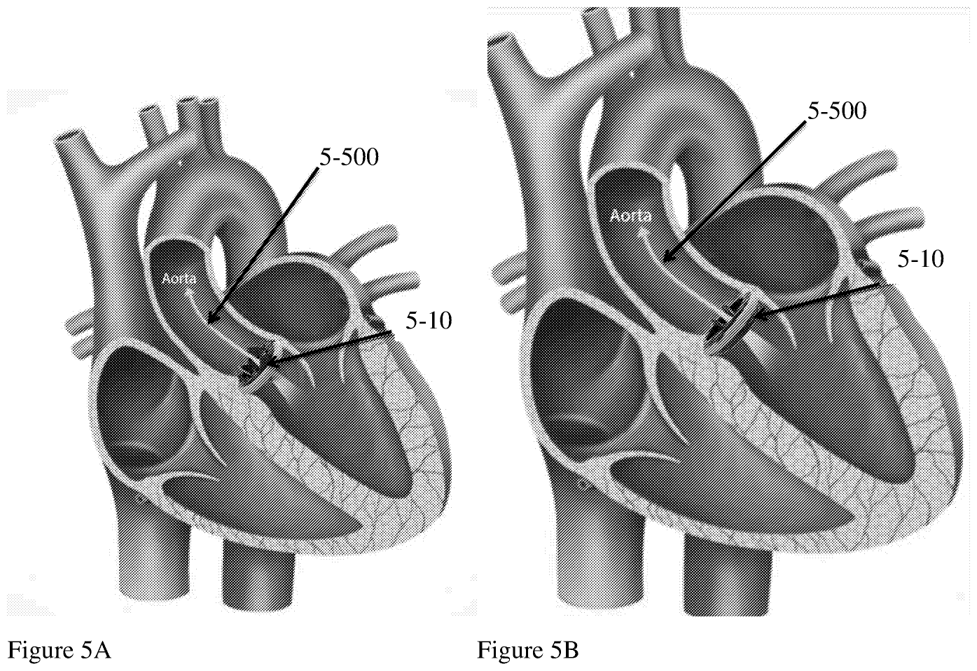

FIGS. 4A-4D depict bi-leaflet valves, whereby an internal drainage port 4-400, which vents any fluid accumulating therein to a region external to the valve is depicted, for example, via a further egress channel 4-410 to that blood does not pool within internal regions of the valve devices. Also depicted are securing means 4-420, which secure embodied components of the constraining ring, which may in some aspects thread into a cognate housing in the constraining ring. In some aspects, the constraining ring is comprised of a lower ring, 4-510, which seamlessly connects to an upper ring 4-520, via securing means 4-420 attaching to cognate securing housings 4-430 in at least one element of the ring, whereby the hinge regions 4-500 of the leaflets 4-20 are simultaneously well and seamlessly secured in the constraining ring, including in some embodiments, via a securable cover 4-440 which specifically attaches over the hinge region.

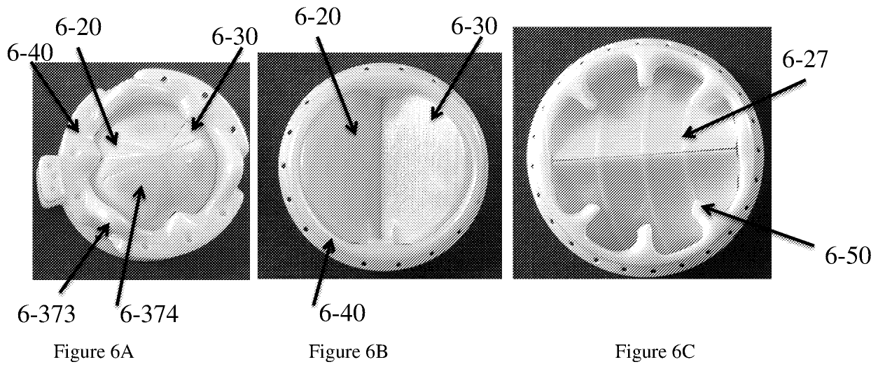

FIGS. 5A-5B depict arrangement of a blood flow modulating ring device 5-10 in tri-leaflet and bi-leaflet forms, respectively, whereby the ring is placed preceding or following the valve with respect to the flow path of blood 5-500 through the valve device and FIGS. 6A-6C present photographs of valve devices produced as described in the Examples hereinunder, which represent further non-limiting examples of envisioned valve devices for use in accordance with the provisions of this invention.

The invention having been described, the following examples are intended to illustrate, and not limit, the invention.

### EXAMPLES

### Example 1

### Preparation of a Composite Valve Device and/or Modular Composite Valve Assembly of this Invention

Preparation of the valve devices of this invention may be accomplished via standard methodology. For example, and as a non-limiting embodiment, the components of the valve devices as herein described and depicted may be prepared by standard injection methods, for example, making use of a tungsten carbide injector machine. In some aspects, the devices of this invention that may comprise steel or nitinol components, which in turn may be prepared using a spring production process. In some aspects, such elements produced thereby may be attached to a different component, e.g. to the base by laser welding or spot welding or other similar means. Coatings may be applied to an underlying structure via known methods, as well. In some aspects, the coating may include a diamond-like coating (DLC) as is known in the art, and in some embodiments, the coating may include physical vapor deposited (PVD's) coatings.

In some embodiments, the coatings may comprise a DLC coating, a Co—Cr composite, a Nano-thin film such as Nano-glass and CK1 ceramic, Nano-stell FM01 and NFM02 and the like, as will be appreciated by the skilled artisan. In some aspects, the valve systems of this invention may comprise parts made of different materials, such as, for example, a valve constraining ring comprised of a first material, for example titanium, which in turn may be coated with a first substance, and leaflets comprised of a different material, for example, a ceramic material, which may further comprise a DLC oprional coating.

Polishing may be accomplished via methods well recognized in the art.

### Example 2

### Incorporation of a Composite Valve Device and/or Modular Composite Valve Assembly of this Invention

A valve device is prepared, for example in accordance with the procedures broadly described in Example 1. Such devices are then assessed via standard methodology demonstrating marked improvement via the use of the embodied devices of this invention in comparison to conventional valve devices.

In vivo assessment of the valve devices can also be conducted in a survival surgery setting, using animal models, as will be appreciated by the skilled artisan. The improved functionality as a result of the procedure can be assessed using standard evaluation means, for example, valve competence and functionality may be assessed by Echo (TTE/TEE) evaluation (intra/post operatively, shorter X clamp/pump time resulting in better outcome parameters (less intraoperative bleeding, shorter hospitalization time, etc).

Clinical trials to evaluate the devices are also envisioned.

### Example 3

### Preparation of a Valve Device of this Invention

FIGS. 6A-6C depict valves prepared in accordance with the principles of this invention and represent an embodied aspect of same.

In each case a rough tungsten carbide mold was made in a shape approximating that of the desirable final product. The following ceramic compounds: SiC—Silicon Carbide, B4C—Boron Carbide, Al2O3—Alumina, ZTA—Zirconia and Alumina compounds were evaluated, using the following percentages:

For the leaflets: 3-4.5, for the ring: 4.2-5.6, depending on the material used with Boron Carbide being the lightest, and Alumina being the heaviest, grams of ceramic compound powder were inserted into a the mold and then pressed with a force of 4-8 tons to form the desired shaped disc.

The disc was then subjected to initial sintering, where it is heated to 500 degrees Celsius in gradual intervals for a time of 6-8 hours. Following the initial sintering, the shaped ceramic object, or “green body”, was machined to form its final desired shape and then subjected to final sintering, which was initiated at 500 degrees Celsius and gradually reached 1000-1500 degrees over an 18-24 hour timeframe, heated in increasing intervals of approximately 100 degrees each time. Following final sintering, the object was then further smoothed through known methods, and parts were assembled to create the final product.

In some aspects, the composite valve system will comprise one or more magnets located in the leaflets and/or in the constraining ring, which in some embodiments, is prepared by including same in the mold when the e.g. pressing of the ceramic powder is accomplished, followed by the sintering process described herein. In some aspects, the magnets may be incorporated with a strong adhesive to increase the association of same with the valves, and in some embodiments, a compartment is created within the leaflets and ring, such as, for example, as depicted in FIGS. 3A-3F, where the magnets d-375 and d-376 are inserted in hollowed compartments such as, d-385, which compartments are then sealed with conventional means, for example via filling with an appropriate biocompatible resin or other suitable material and the cover applied 3-374.

In some aspects the valves are secured in place with any appropriate suture or pins, which is not meant to be limiting in terms of the types of securing mechanisms and parts envisioned for use in this context.

In a further embodied method of preparing the valves of this invention, the valve system may be created as a uniform single solid piece construction and in some embodiments, the valve systems of this invention may be created in a series of parts seamlessly joined together. In some embodiments, the ceramic material is subjected to compression, polishing, and introduced via catheterization with further in situ assembly.

In one aspect, a three-dimensional model of the valve systems is constructed, defining the cylinders, tolerance permitted about an axis, angle of planes of parts, thickness of individual components, leaflets and designs predicting desired flow patterns are devised, and in some embodiments, internal and external diameters/widths and lengths of the component parts and/or flow rates being accomodated are designed in consideration of the overall dimensions in the patient into which the systems will be inserted and/or in consideration of the valve being replaced or aided with the systems of this invention.

In one embodied method, a three-dimensional model of the valve system as depicted in FIG. 6A-6C may be prepared using the Solid works suite of software.

The model is then scaled and printed using a 3-D printer in accordance with manufacturer's instructions, in further consideration of the Solid Works model regarding same.

It is noted that, as will be appreciated by the skilled artisan, scaling will require revising values for printing different parts and using different materials, as dependent upon the particular application/system being devised.

As will be appreciated certain hinge regions or regions permitting rotation of a part about an axis are present in the valve systems of the invention, for example, allowing rotation of the leaflets from closed-to-open configurations. Certain hollowed parts are necessary in the valve systems as herein described as well, for example, to accommodate suturing therethrough and/or drainage therethrough, and other applications, as will be appreciated by the skilled artisan.

In some aspects, such regions requiring the creation of space at desired regions in the solid parts of the valve systems, any number of methods may be used to create a desired region devoid of the solid materials, for example, by cutting, drilling, or mold creation of the component parts to create regions devoid of the solid materials.

In other aspects, and representing contemplated embodied methods herein, a binder or manipulatable chemical composition is deposited in the desired region, devoid of the ceramic material being printed, for example, by patterning an acrylic resin, or other composition that can deposit in the desired region, and ultimately be site specifically removed, whereas the ceramic material, for example, as prepared in the instant example, using zirconium oxide, or in another prepared version, where silicon carbide was used, and either ceramic material remains.

For example, and in a representative, non-limiting example, an acrylic resin may be deposited at a site of desired creation of a void/hollowed area, which when the printed ceramic valve is exposed to temperatures of above a defined temperature, will result in evaporation of the composition, thereby creating the hollow/void in the desired region.

In another representative, non-limiting example, a solvent may be applied, which selectively dissolves the manipulatable chemical composition deposited whose dissolution is desired in distinct regions.

According to this aspect, and in exemplified versions prepared according to this example, both silicon carbide and/or zirconium oxide ceramic material was deposited in accordance with the 3-dimensional model prepared, and scaled, and expoxy resin was printed instead of the ceramic materials at regions which constitute ultimately regions that are hollowed in the final product.

Following exposure of the printed product to temperatures spanning 120-200 degree Celsius, the resin preferentially evaporated, leaving a hollowed area at the desired region.

In one exemplified aspect, following printing the printed product underwent polishing, then was fired using a gradually increasing temperature up to 1500 degrees, for a time suitable to the scaled size of the product. The fired valve system was then selectively, gradually cooled, to room temperature and the product underwent a second polishing and assembly.

### Example 4

### Hemodynamity & Thrombogenicity of Novel Aortic Mechanical Valve Implant

### Materials and Methods:

Sheep (ASAF breed) 84 kg originating from an approved dealer of the Technion PCRA-(with known herd health status) B.C. & Chemistry were purchased and were anesthetized in accordance with institutional protocols. Briefly, 800 mg ketamine (10 mg/kg)+Xylazine 0.2 cc (0.05 mg/kg) was administered. The induction was accomplished with the use of Propofol—10 cc (3-6 mg/kg) and maintenance was accomplished with Isoflurane 1-3%+Fentanyl 3-5 mcg/kg/hr/IV

Following anesthesia the sheep was continuously monitored for their of I.B.P, ECG, ETCo2, T°, and O2 values.

A Foley catheter was placed in the urinary bladder, and sheaths inserted into an ear artery and a central vein to enable monitoring and fluid treatment.

The sheep was placed in lateral recumbence and after sterile preparation a left thoracotomy between the 4-5th intercostal enabled approach to the thoracic descending Aorta. Heparin was administered to double ACT baseline for the duration of the procedure. Cross clamps were placed on proximal and distal location of graft implantation, enabling dissection and implantation of the 23 mm valve as depicted in FIG. 6B, placed within a 26 mm Vascutek graft.

At the time of cross clamp application no evidence of ischemia to hind legs was evident.

After chest closure under fluoroscopy the valve appeared stable, valve leaflets were fixed and no movement was evident, given the valve placement was to demonstrate biocompatibility, however, full & fast flow of contrast media was evident through the valve.

The animal was rehabilitated and returned to its pen.

1. A valve device, comprising:
two or more leaflets each comprising:
at least a first material which reduces electrostatic forces between blood and device components, reduces adhesion between blood and device components or a combination thereof; and
optionally at least a first magnet incorporated within at least one of said leaflets; and
a valve constraining ring, comprising:
at least a first material which reduces electrostatic forces between blood and device components, reduces adhesion between blood and device components or a combination thereof; and
internally located attachment regions for said two or more leaflets; and
optionally at least a second magnet of matched polarity to said first magnet, incorporated within the valve constraining ring at regions proximal to said first magnet within said leaflet such that repulsive forces are present when said first magnet and said second magnet are brought into proximity with each other in the absence of flow; and
one or more extending projections, which reduce turbulent flow through the device, or proximally thereto, which are located on the leaflets of the valve device, or which are located on the constraining ring component of the device or a combination thereof.

2. The valve device of claim 1, wherein said valve is comprised of a composite of a first material and a second material.

3. The valve device of claim 2, wherein said first material and said second material are located on the same surface exposed regions of said two or more leaflets, said valve constraining ring, or a combination thereof, whereby said first material is a more apically located layer and said second material is a more basally located layer.

4. The valve device of claim 2, wherein said first material and said second material are located on the same surface exposed regions of said two or more leaflets, said valve constraining ring, or a combination thereof, whereby said first material is a more basally located layer and said second material is a more apically located layer.

5. The valve device of claim 2, wherein said first material and said second material are located on different surface exposed regions of said two or more leaflets, said valve constraining ring, or a combination thereof.

6. The valve device of claim 2, wherein said two or more leaflets comprise said first material on a first surface of the leaflet and said second material on an opposing surface of the leaflet.

7. The valve device of claim 2, wherein said two or more leaflets comprise said first material on a first surface of said leaflet and a second of said two or more leaflets comprise said second material on a first surface of said leaflet.

8. The valve device of claim 7, wherein a first of said two or more leaflets comprise said second material on a second surface of said leaflet and a second of said two or more leaflets comprise said first material on a second surface of said leaflet.

9. The valve device of claim 1, wherein said two or more leaflets, said valve constraining ring, or a combination thereof is comprised of an at least semi-flexible material.

10. The valve device of claim 1, wherein said two or more leaflets, said valve constraining ring, or a combination thereof is comprised of at least a first region that is flexible and a second region that is more rigid than said first region.

11. The valve device of claim 1, wherein said first material is a ceramic material, or comprises a layer of ceramic material.

12. The valve device of claim 1, wherein said two or more leaflets, said valve constraining ring, or a combination thereof is comprised of a metal, a polymeric material, or a combination or composite thereof.

13. The valve device of claim 1, wherein said two or more leaflets, said valve constraining ring, or a combination thereof is comprised of nitinol, stainless steel, PTFE, polyester, silicon or a combination or composite thereof.

14. The valve device of claim 1, wherein said two or more leaflets, said valve constraining ring, or a combination thereof is comprised of silicon nitride, silicon carbide, titanium nitride, or pyrolytic carbon (PyC) or combinations thereof.

15. The valve device of claim 1, wherein said two or more leaflets, said valve constraining ring, or a combination thereof is comprised of Zirconia toughened alumina (ZTA), boron carbide, nanocrystalline zirconium oxide or combinations thereof.

16. The valve device of claim 1, wherein said two or more leaflets, said valve constraining ring, or a combination thereof further comprises a coating.

17. The valve device of claim 1, wherein said first magnet, said second magnet or a combination thereof comprises Neodymium coated with a biocompatible polymer, silicon nitride, silicon carbide, titanium nitride, or pyrolytic carbon (PyC).

18. The valve device of claim 1, further comprising at least a third magnet incorporated within said leaflet structure, which third magnet is weaker in terms of its strength and opposite in terms of its polarity as compared to said first magnet.

19. The valve device of claim 18, wherein said third magnet is incorporated within the same leaflet as said first magnet.

20. The valve device of claim 18, wherein said third magnet is incorporated within a different leaflet as said first magnet.

21. The valve device of claim 1, further comprising at least one externally located ratcheting locking mechanism is constructed such that advancement of a suture-like structure therethrough is uni-directional and regulated.

22. A method of performing cardiac valve repair or replacement surgery on a patient comprising the steps of applying the valve device of any one of claims 1-21 to replace a defective or malfunctioning valve in a subject.
